(19) United States
(12) Patent Application Publication
(10) Pub. No.: US 2013/0281696 A1
(43) Pub. Date: Oct. 24, 2013

(54) PROCESS FOR PREPARING BRANCHED ALCOHOLS

(71) Applicant: BASF SE, Ludwigshafen (DE)

(72) Inventors: Thomas SCHAUB, Neustadt (DE); Pepa Dimitrova, Worms (DE); Rocco Paciello, Bad Duerkheim (DE); Frederic Bauer, Deidesheim (DE)

(73) Assignee: BASF SE, Ludwigshafen (DE)

(21) Appl. No.: 13/864,774

(22) Filed: Apr. 17, 2013

Related U.S. Application Data
(60) Provisional application No. 61/635,897, filed on Apr. 20, 2012.

(51) Int. Cl. C07C 29/32; B01J 31/22

(52) U.S. Cl. CPC C07C 29/32; B01J 31/2295
USPC 546/2; 568/902

(57) ABSTRACT

Process for preparing branched alcohols of the general formula (I)

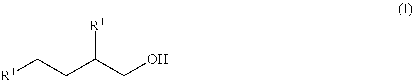

where the groups R¹ are different or identical and selected from C₂-C₃-alkyl, linear or branched, using at least one alcohol of the formula (II)

R¹—CH₂—CH₂—OH (II)
in homogeneous phase
in the presence of at least one base,
   wherein at least one Ru(II)-containing complex compound is used in which the Ru(II) has at least one ligand L¹ which is at least bidentate, where at least one coordination site of L¹ is a nitrogen atom.

The present invention relates to a process for preparing branched alcohols of the general formula (I)

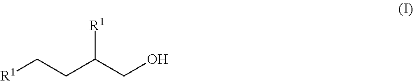

where the groups R¹ are different or identical and selected from C₂-C₃-alkyl, linear or branched, using at least one alcohol of the formula (II)

R¹—CH₂—CH₂—OH (II)

in homogeneous phase
in the presence of at least one base,
wherein at least one Ru(II)-containing complex compound is used in which the Ru(II) has at least one ligand L¹ which is at least bidentate, where at least one coordination site of L¹ is a nitrogen atom.

Branched fatty alcohols find diverse uses as intermediates, for example for producing surfactants. It is therefore of interest to develop economical processes for preparing branched fatty alcohols and in particular fatty alcohols branched in the 2 position. Of particular interest in this connection are processes for preparing Guerbet alcohols which have further branches besides the branching in position 2.

It is known from J. Org. Chem. 2006, 71, 8306 that Guerbet reactions can be catalyzed with the help of iridium complexes. Specifically, it is known from the cited passage that with the help of [Cp*IrCl₂]₂ and 1,7-octadiene and potassium tert-butanolate in p-xylene as solvent it is possible to dimerize the branched alcohol isoamyl alcohol to give the corresponding Guerbet alcohol (Cp*: pentamethylcyclopentadienyl). However, the yield is not optimal, and iridium compounds are expensive.

U.S. Pat. No. 3,514,493 discloses the preparation of 2-ethylhexanol and of 2-butyloctanol with the help of supported metals, for example palladium or ruthenium supported on activated carbon. J. Organomet. Chem. 1972, 37, 385 proposes that Guerbet alcohols can be made with the help of RuCl₃ with certain phosphane ligands in homogeneous phase.

However, the use of the catalyst indicated in J. Organomet. Chem. 1972, 37, 385 for preparing Guerbet alcohols which have further or other branches as well as the branching in position 2 has not been successful. In particular, the attempt to prepare a Guerbet alcohol on the basis of so-called “biobased isoamyl alcohols” produced from fusel oils is not possible.

It was therefore the object to provide a versatile process with the help of which it is possible to also prepare those Guerbet alcohols which have further or other branches as well as branching in position 2. It was also the object to provide catalysts which are suitable for preparing Guerbet alcohols and in particular those Guerbet alcohols which have further or other branches as well as the branching in position 2.

Accordingly, the process defined at the start has been found, also called process according to the invention for short.

The process according to the invention proceeds from at least one alcohol of the general formula (II)

R¹—CH₂—CH₂—OH (II)

in which R¹ is selected from C₂-C₃-alkyl, linear or—if possible—branched, e.g. ethyl, n-propyl and isopropyl, preferably ethyl and isopropyl, very particularly preferably isopropyl.

Alcohol of the general formula (II) can be used in pure form or in the form of mixtures, in particular in the form of isomeric mixtures, in particular in the form of mixtures with at least one isomeric alcohol. In this connection, in the case of R¹=propyl, the isomeric alcohol(s) can correspond to formula (II). In a particular variant of the present invention, alcohol of the general formula (II) is used in a mixture with at least one such isomeric alcohol which does not correspond to formula (II).

An example of a suitable isomeric alcohol of isoamyl alcohol (R¹=isopropyl) is 2-methylbutanol. This reacts with isoamyl alcohol preferably to give an alcohol of the formula (Ia)

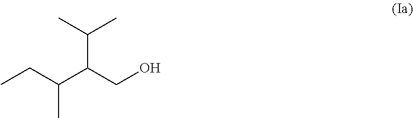

In one embodiment of the present invention, alcohol of the formula (II) is used in a mixture with 0.1 to 25 mol % of at least one isomeric alcohol which can, but preferably does not, correspond to formula (II).

Alcohol of the general formula (II) and in particular mixtures of alcohol of the general formula (II) with one or more of its isomers can be prepared by synthesis or on the basis of biological raw materials, for example by fermentation or other biological degradation of saccharides.

The process according to the invention is carried out in a homogeneous phase, i.e. the catalyst is not used in a form deposited on a solid support and no emulsion is produced in which the reactants react with one another. However, within the context of the present invention, it is entirely possible that alcohol of the general formula (II) or at least one of its isomers are present at least partially in the gas phase.

The catalyst or catalysts here are dissolved completely or at least predominantly in the reaction mixture, for example to at least 90 mol %, preferably to at least 95 mol %, based on Ru(II).

The process according to the invention can be carried out in the presence of at least one solvent which is different from alcohol of the general formula (II), for example in the presence of aromatic solvents such as, for example, para-xylene, ortho-xylene, meta-xylene, isomer mixtures of xylene, mesitylene, or in the presence of toluene, ethylbenzene or of aliphatic or cycloaliphatic solvents such as, for example, n-hexane, n-heptane, n-octane, n-nonane, n-dodecane or decalin. It is preferred to carry out the process according to the invention without adding solvent which is different from alcohol of the general formula (II).

The process according to the invention is carried out in the presence of at least one catalyst which can be prepared before the actual Guerbet reaction or preferably in situ while carrying out the process according to the invention. The catalyst used is at least one Ru(II)-containing complex compound in which the Ru(II) has at least one ligand L¹ which is at least bidentate, preferably bidentate or tridentate, where at least one coordination site of L¹ is a nitrogen atom. Within the context of the present invention, ligands of this type are also referred to for short as “L¹” or “ligand L¹”.

In one embodiment of the present invention, ligand L¹ is coordinated with Ru(II) via two, three or four nitrogen atoms, preferably via two or three, and L¹ has no coordination sites different from nitrogen. An example of a bidentate ligand L¹ which coordinates with Ru(II) via two nitrogen atoms and has no coordination sites different from nitrogen is 2,2′-bipyridyl.

In another embodiment of the present invention, ligand L¹ is coordinated with Ru(II) via two or three coordination sites, of which one or two coordination site(s) is/are different from nitrogen and the other(s) is/are nitrogen atom(s). Coordination sites of ligand L¹ different from nitrogen are selected from phosphorus atoms, oxygen atoms, sulfur atoms and in particular carbene carbon atoms.

Nitrogen atoms which coordinate to Ru(II) are preferably selected here from tertiary amine nitrogen atoms which are part of a heterocycle', and nitrogen atoms which are part of a tertiary amino group which is not part of a heterocycle'.

In one embodiment of the present invention, L¹ is selected from compounds of the general formula (III)

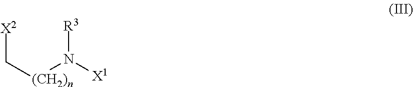

where the variables are selected as follows:
R³ is selected from
hydrogen,
C₁-C₁₀-alkyl, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; preferably n-C₁-C₄-alkyl such as methyl, ethyl, n-propyl, n-butyl, in particular methyl;
C₃-C₁₀-cycloalkyl, for example cyclopropyl, cyclobutyl, cyclopentyl, cyclohexyl, cycloheptyl, cyclooctyl, cyclononyl and cyclodecyl, preferably C₅-C₇-cycloalkyl, in each case unsubstituted or mono- or polysubstituted, for example with methyl, methoxy or ethyl,
   n is selected from zero and one,
   X¹ is selected from
hydrogen,
C₁-C₅-alkyl, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, in particular methyl or isopropyl, and
(CH₂)ₙ₊₁—X²,
   X² is selected from NR⁴R⁵, 2-pyridyl and imidazol-2-ylidenyl of the formula

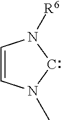

R⁴, R⁵ are different or preferably identical and selected from
C₁-C₁₀-alkyl, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; preferably tert-butyl or n-C₁-C₄-alkyl such as methyl, ethyl, n-propyl, n-butyl, in particular tert-butyl or methyl;
C₃-C₁₀-cycloalkyl, for example cyclopropyl, cyclobutyl, cyclopentyl, cyclohexyl, cycloheptyl, cyclooctyl, cyclononyl and cyclodecyl, preferably C₅-C₇-cycloalkyl, in each case unsubstituted or mono- or polysubstituted, for example with methyl, methoxy or ethyl,
benzyl and
phenyl, unsubstituted or mono- or polysubstituted with C₁-C₃-alkyl, for example para-methylphenyl, para-ethylphenyl, 2,6-dimethylphenyl, 2,4,6-trimethylphenyl, 2,6-diethylphenyl, 2,6-diisopropylphenyl and 2-methyl-4-isopropylphenyl,
R⁶ is selected from C₁-C₁₀-alkyl, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; preferably n-C₁-C₄-alkyl such as methyl, ethyl, n-propyl, n-butyl, in particular methyl;
C₃-C₁₀-cycloalkyl, for example cyclopropyl, cyclobutyl, cyclopentyl, cyclohexyl, cycloheptyl, cyclooctyl, cyclononyl and cyclodecyl, preferably C₅-C₇-cycloalkyl, in each case unsubstituted or mono- or polysubstituted, for example with methyl, methoxy or ethyl,
benzyl and
phenyl, unsubstituted or mono- or polysubstituted with C₁-C₃-alkyl, for example para-methylphenyl, para-ethylphenyl, 2,6-dimethylphenyl, 2,4,6-trimethylphenyl, 2,6-diethylphenyl, 2,6-diisopropylphenyl and 2-methyl-4-isopropylphenyl.

In one embodiment of the present invention, L¹ is selected from compounds of the general formula (IV)

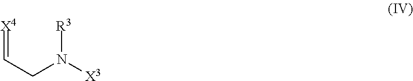

where the variables are selected as follows:
R³ is selected from
hydrogen,
C₁-C₁₀-alkyl, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; preferably n-C₁-C₄-alkyl such as methyl, ethyl, n-propyl, n-butyl, in particular methyl;
C₃-C₁₀-cycloalkyl, for example cyclopropyl, cyclobutyl, cyclopentyl, cyclohexyl, cycloheptyl, cyclooctyl, cyclononyl and cyclodecyl, preferably C₅-C₇-cycloalkyl, in each case unsubstituted or mono- or polysubstituted, for example with methyl, methoxy or ethyl,
   benzyl and
phenyl, unsubstituted or mono- or polysubstituted with C₁-C₃-alkyl, for example para-methylphenyl, para-ethylphenyl, 2,6-dimethylphenyl, 2,4,6-trimethylphenyl, 2,6-diethylphenyl, 2,6-diisopropylphenyl and 2-methyl-4-isopropylphenyl.
   X³ is selected from
hydrogen,
C₁-C₅-alkyl, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl; preferably n-C₁-C₄-alkyl such as methyl, ethyl, n-propyl, n-butyl, in particular methyl, and
CH₂═X⁴,
   X⁴ is selected from NR⁴, and
   R⁴ is selected from
C₁-C₁₀-alkyl, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl; preferably tert-butyl or n-C₁-C₄-alkyl such as methyl, ethyl, n-propyl, n-butyl, in particular methyl or tert-butyl,
C₃-C₁₀-cycloalkyl, for example cyclopropyl, cyclobutyl, cyclopentyl, cyclohexyl, cycloheptyl, cyclooctyl, cyclononyl and cyclodecyl, preferably C₅-C₇-cycloalkyl, in each case unsubstituted or mono- or polysubstituted, for example with methyl, methoxy or ethyl,
benzyl and
phenyl, unsubstituted or mono- or polysubstituted with C₁-C₃-alkyl, for example para-methylphenyl, para-ethylphenyl, 2,6-dimethylphenyl, 2,4,6-trimethylphenyl, 2,6-diethylphenyl, 2,6-diisopropylphenyl and 2-methyl-4-isopropylphenyl.

In one embodiment of the present invention, L¹ is selected from compounds of the general formula (V)

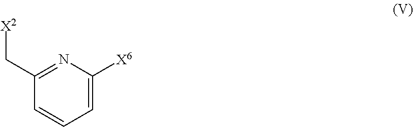

where the variables are selected as follows:
X⁶ is selected from hydrogen,
C₁-C₅-alkyl, for example methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl; preferably n-C₁-C₄-alkyl such as methyl, ethyl, n-propyl, n-butyl, in particular methyl, and
CH₂—X²,
   X² is selected from NR⁴R⁵, 2-pyridyl and imidazol-2-ylidenyl of the formula

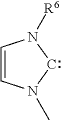

R⁴, R⁵ are different or preferably identical and selected from C₁-C₁₀-alkyl, C₃-C₁₀-cycloalkyl, benzyl and phenyl, unsubstituted or mono- or polysubstituted with C₁-C₃-alkyl,
R⁶ is selected from C₁-C₁₀-alkyl, C₃-C₁₀-cycloalkyl, benzyl and phenyl, unsubstituted or mono- or polysubstituted with C₁-C₃-alkyl.

R³, R⁴, R⁵ and R⁶ are as defined in more detail above.

In one embodiment of the present invention, L¹ is selected from compounds of the general formula (VI)

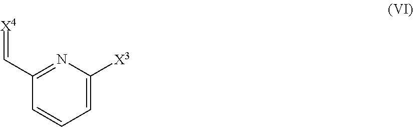

where the variables are selected as follows:
X³ is selected from hydrogen, C₁-C₅-alkyl and CH₂═X⁴,
X⁴ is identical or optionally different, preferably identical, and selected from N—R⁴,
R⁴ is C₁-C₁₀-alkyl, C₃-C₈-cycloalkyl, benzyl or phenyl, unsubstituted or mono- or polysubstituted with C₁-C₃-alkyl.

In one embodiment of the present invention, L¹ is selected from compounds of the general formula (VII)

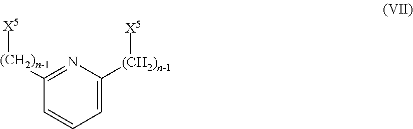

where the variables are selected as follows:
n is different or preferably identical and in each case zero or one
X⁵ is in each case identical and selected from NR⁴R⁵, 2-pyridyl and imidazol-2-ylidenyl of the formula

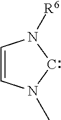

R⁴, R⁵ are different or identical and selected from hydrogen, C₁-C₁₀-alkyl, C₃-C₁₀-cycloalkyl, benzyl and phenyl, unsubstituted or mono- or polysubstituted with C₁-C₃-alkyl,
R⁶ is selected from C₁-C₁₀-alkyl, C₃-C₁₀-cycloalkyl, benzyl and phenyl, unsubstituted or mono- or polysubstituted with C₁-C₃-alkyl.

R³, R⁴, R⁵ and R⁶ are as defined in more detail above.

Particularly preferred examples of ligands L¹ are those of the formula (VII.1)

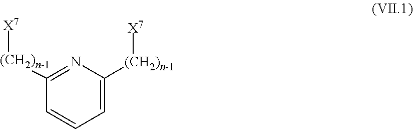

in which the groups (CH₂)ₙ₋₁—X⁷ are in each case identical and selected from
2-pyridyl (i.e. in each case n=zero),
CH₂—N(CH₃)₂, CH₂—N(C₂H₅)₂, CH₂—N(n-C₃H₇)₂, CH₂—N(n-C₄H₉)₂, CH₂—N(iso-C₃H₇)₂, CH₂—N(tert-C₄H₉)₂, CH₂—N(n-C₅H₁₁)₂, CH₂—N(n-C₆H₁₃)₂, CH₂—N(n-C₈H₁₇)₂, CH₂—N(C₆H₅)₂, CH₂—N(CH₂—C₆H₅)₂, and CH₂—N(cyclo-C₆H₁₁)₂, (i.e. in each case n=1), and

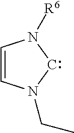

(i.e. in each case n=1), where R⁷ is selected from
methyl, ethyl, n-propyl, isopropyl, n-butyl, n-pentyl, n-hexyl, n-heptyl, n-octyl, preferably isopropyl or n-C₁-C₄-alkyl such as methyl, ethyl, n-propyl, n-butyl, in particular methyl,
cyclohexyl and
phenyl, unsubstituted or mono- or up to trisubstituted with identical or different C₁-C₃-alkyl, for example para-methylphenyl, 2,6-dimethylphenyl, 2,4,6-trimethylphenyl, 2,6-diethylphenyl, 2,6-diisopropylphenyl and 2-methyl-4-isopropylphenyl.

Other particularly preferred examples of ligands L¹ are those of the formula (VI.1)

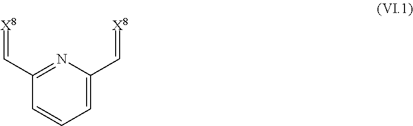

in which X⁸ are in each case identical and selected from N—CH₃, N—C₂H₅, N-n-C₃H₇, N-n-C₄H₉, N-iso-C₃H₇, N-n-C₅H₁₁, N-n-C₆H₁₃, N-n-C₅H₁₇, N—CH₂—C₆H₅ and N-cyclo-C₆H₁₁, and
N-phenyl, unsubstituted or mono- or up to trisubstituted with identical or different C₁-C₃-alkyl, for example N-para-methylphenyl, N-2,6-dimethylphenyl, N-2,4,6-trimethylphenyl, N-2,6-diethylphenyl, N-2,6-diisopropylphenyl and N-(2-methyl-4-isopropylphenyl).

A very particularly preferred ligand L¹ is 2,6-bis-2-pyridylpyridine, within the context of the present invention also called “terpyridyl” for short.

In one embodiment of the present invention, Ru(II)-containing complex compound can have at least one further ligand selected from CO, pseudohalides, organic carbonyl compounds, aromatics, olefins, phosphanes, hydride and halides.

Here, “at least one further ligand” is to be understood as meaning a ligand which is different from ligand L¹. Examples of further ligands are
CO (carbon monoxide),
pseudohalide, in particular cyanide, isocyanate and rhodanine,
organic carbonyl compounds, for example ketones, preferably organic dicarbonyl compounds such as acetyl acetonate, 1-phenylbutane-1,3-dione, acetic ester
aromatics which may be electrically charged or uncharged. Preferred examples of uncharged aromatics are benzene, toluene, para-xylene, hexamethylbenzene and para-cymene. Preferred examples of electrically charged aromatics are negatively charged aromatics, in particular cyclopentadienyl, indenyl, 4,5-benzindenyl and Cp* (pentamethylcyclopentadienyl),
olefins, electrically neutral or as anions, for example COD (1,5-cyclooctadienyl), allyl or methallyl (2-methylallyl),
phosphanes, for example mono-, di- or triphosphanes, preferably monophosphanes, in particular tertiary aromatic phosphanes, for example triphenylphosphane,
hydride and
halogens, for example bromide and in particular chloride.

Examples of phosphanes suitable as further ligand are those which have at least one unbranched or branched C₁-C₁₂-alkyl radical, at least one C₃-C₁₂-cycloalkyl radical or at least one aromatic radical having up to 24 carbon atoms. Examples of C₁-C₁₂-alkyl radicals are methyl, ethyl, 1-propyl, 2-propyl, 1-butyl, 2-butyl, 1-(2-methyl)propyl, 2-(2-methyl)propyl, 1-pentyl, 1-hexyl, 1-heptyl, 1-octyl, 1-nonyl, 1-decyl, 1-undecyl, 1-dodecyl, 1-(2-methyl)pentyl, 1-(2-ethyl)hexyl, 1-(2-n-propyl)heptyl. Preferred C₁-C₁₂-alkyl radicals are selected from ethyl, 1-butyl, sec-butyl and 1-octyl.

Examples of C₃-C₁₂-cycloalkyl radicals are in particular selected from C₄-C₈-cycloalkyl radicals, branched or unbranched, such as cyclopentyl, cyclohexyl, cycloheptyl, cyclooctyl, methylcyclopentyl, for example 2-methylcyclopentyl, 3-methylcyclopentyl, also 2,5-dimethylcyclopentyl (syn, anti or as isomer mixture), 2-methylcyclohexyl, 3-methylcyclohexyl, 4-methylcyclohexyl, 2,6-dimethylcyclohexyl (syn, anti or as isomer mixture), norbonyl and —CH₂—C₆H₁₁. A preferred C₃-C₁₂-cycloalkyl radical is cyclohexyl.

In a preferred variant, the further ligand selected is a phosphane which carries two, particularly preferably three identical radicals, for example tri-n-butylphosphane, tri-sec-butylphosphane, tricyclohexylphosphane or tri-n-octylphosphane.

In one embodiment, the substituents of phosphane suitable as a further ligand that are selected are at least one aromatic radical, for example 9-anthracenyl, preferably three identical aromatic radicals, for example phenyl, 2-tolyl, 3-tolyl, para-tolyl, xylyl, 1-naphthyl, 2-naphthyl, 1-binaphthyl, para-anisyl, 2-ethylphenyl, 3-ethylphenyl, para-ethylphenyl, 2-chlorophenyl, para-chlorophenyl, 2,6-dichlorophenyl, or at least one heteroaromatic radical. Examples of heteroaromatic radicals are thienyl, benzothienyl, 1-naphthothienyl, thianthrenyl, furyl, benzofuryl, pyrrolyl, imidazolyl, pyrazolyl, pyridyl, pyrazinyl, pyrimidinyl, pyridazinyl, indolyl, isoindolyl, indazolyl, purinyl, isoquinolinyl, quinolinyl, acridinyl, naphthyridinyl, quinoxalinyl, quinazolinyl, cinnolinyl, piperidinyl, carbolinyl, thiazolyl, oxazolyl, isothiazolyl, isoxazolyl. The heteroaryl groups can be unsubstituted or substituted with one or more substituents which are defined above under C₁-C₁₂-alkyl.

In another preferred variant, the further ligand selected is a polydentate phosphane, for example with the grouping>P—CH₂—CH₂—P(C₁-C₁₀-alkyl)-CH₂CH₂—P<, particularly preferably with the grouping>P—CH₂—CH₂—P<. An example is 1,2-bis(dicyclohexylphosphino)ethane.

In one embodiment of the present invention, 0.001 to 5 mol % of Ru(II) are used, based on alcohol of the general formula (II).

The process according to the invention is carried out in the presence of at least one base. Preferred bases are Brönsted bases. Examples of suitable bases which may be mentioned are: LiOH, NaOH, KOH, LiH, NaH, KH, Ca(OH)₂, CaH₂, LiAlH₄, NaBH₄, LiBH₄, Na₂CO₃, NaHCO₃, Li₂CO₃, LiHCO₃, K₂CO₃, KHCO₃, K₃PO₄, Na₃PO₄, n-butyllithium, tert-BuLi, methyllithium, phenyllithium, lithium methanolate, lithium ethanolate, LiO-n-C₃H₇, LiO-iso-C₃H₇, LiO-n-C₄H₉, LiO-iso-C₄H₉, LiO-n-C₅H₁₁, LiO-iso-C₅H₁₁, LiO-n-C₆H₁₃, LiO-iso-C₆H₁₃, lithium n-heptanolate, lithium n-octanolate, lithium benzylate, lithium phenolate, potassium methanolate, potassium ethanolate, KO-n-C₃H₇, KO-iso-C₃H₇, KO-n-C₄H₉, KO-iso-C₄H₉, KO-tert-C₄H₉, KO-n-C₅H₁₁, KO-iso-C₅H₁₁, KO-n-C₆H₁₃, KO-iso-C₆H₁₃, potassium n-heptanolate, potassium n-octanolate, potassium benzylate, potassium phenolate, sodium methanolate, sodium ethanolate, NaO-n-C₃H₇, NaO-iso-C₃H₇, NaO-n-C₄H₉, NaO-iso-C₄H₉, NaO-tert-C₄H₉, NaO-n-C₅H₁₁, NaO-iso-C₅H₁₁, NaO-n-C₆H₁₃, NaO-iso-C₆H₁₃, sodium n-heptanolate, sodium n-octanolate, sodium benzylate, sodium phenolate, KN(SiMe₃)₂, LiN(SiMe₃)₂, NaN(SiMe₃)₂, NH₃ and amines of the formula (R⁸)ₐNH₃₋ₐ, where a is selected from 1, 2 and 3, and R⁸=identical or different and independently of one another unsubstituted or at least monosubstituted C₁-C₁₀-alkyl, (—C₁-C₄-alkyl-P(phenyl)₂), C₃-C₁₀-cycloalkyl, C₃-C₁₀-heterocyclyl, where C₃-C₁₀-heterocyclyl is to be understood as meaning those cyclic groups which have 3 to 10 carbon atoms and at least one heteroatom selected from S, also C₅-C₁₄-aryl or C₅-C₁₀-heteroaryl, where C₅-C₁₀-heteroaryl has at least one heteroatom selected from N, O and S.

In one embodiment of the present invention, in total 0.01 to 50% by weight of base are used, preferably 0.5 to 15% by weight, based on the total alcohol of the formula (II) used.

In one embodiment of the present invention, the reaction medium is liquid at reaction temperature.

In one embodiment of the present invention, the process according to the invention is carried out at a temperature in the range from 80 to 200° C., preferably 100 to 200° C., particularly preferably in the range from 110 to 170° C.

In one embodiment of the present invention, the process according to the invention is carried out in the presence of at least one inert gas. Suitable inert gases are selected from nitrogen and noble gases, in particular argon. In another embodiment of the present invention, the process according to the invention is carried out in the presence of hydrogen. In a further embodiment, the process according to the invention is carried out in the presence of a mixture of hydrogen and at least one inert gas.

In one embodiment of the present invention, the process according to the invention is carried out at a pressure in the range from 0.1 to 5 MPa absolute, which can be the intrinsic pressure of the solvent and/or of the alcohol of the general formula (II) at the reaction temperature and/or the pressure of a gas such as nitrogen, argon or hydrogen. Preferably, the process according to the invention is carried out at a total pressure up to 3 MPa absolute, particularly preferably at a total pressure of from 0.1 to 1 MPa absolute.

For carrying out the process according to the invention, the procedure can involve for example mixing alcohol of the general formula (II) with base and at least one Ru(II)-containing complex compound which has at least one ligand L¹.

In another embodiment of the present invention, the catalyst is generated in situ. This is to be understood as meaning that Ru(II)-containing complex compound which has at least one ligand L¹ is not isolated, but is produced without further work-up by mixing an Ru(II) or Ru(III) starting compound and ligand L¹, for example by mixing Ru(II) or Ru(III) starting compound and ligand L¹ with base and alcohol of the general formula (II), optionally in the presence of a reducing agent.

Suitable Ru(II) and Ru(III) starting compounds are, for example, Ru(p-cymene)Cl₂]₂, [Ru(benzene)Cl_{2]}_{y}, [Ru(CO)₂Cl_{2]}_{y}, where y is in each case in the range from 1 to 1000, [Ru(CO)₃Cl₂]₂, [Ru(COD)(allyl)], RuCl₃.H₂O, [Ru(acetylacetonate)₃], [Ru(DMSO)₄Cl₂], [Ru(cyclopentadienyl)(CO)₂Cl], [Ru(cyclopentadienyl)(CO)₂H], [Ru(cyclopentadienyl)(CO)₂]₂, [Ru(Cp)(CO)₂Cl], [Ru(Cp*)(CO)₂H], [Ru(Cp*)(CO)₂]₂, [Ru(indenyl)(CO)₂Cl], [Ru(indenyl)(CO)₂H], [Ru(indenyl)(CO)₂]₂, ruthenocene, [Ru(COD)Cl₂]₂, [Ru(Cp*)(COD)Cl], [Ru₃(CO)₁₂], [Ru(PPh₃)₄(H)₂], [Ru(PPh₃)₃(Cl)₂], [Ru(PPh₃)₃(CO)(Cl)₂], [Ru(PPh₃)₃(CO)(Cl)(H)], [Ru(PPh₃)₃(CO)(H)₂] and [Ru(cyclooctadienyl)(methylallyl)₂].

Here, Cp* means pentamethylcyclopentadienyl, COD means 1,5-cyclooctadienyl and methylallyl means 2-methylallyl.

Through the selection of the Ru(II) or Ru(III) starting compound it is possible to influence the selection of the further ligand(s).

In one embodiment of the present invention, ligand L¹ and Ru(II) or Ru(III) starting compound can be used in stoichiometric fractions, in each case based on Ru(II) or Ru(III). In another variant, an excess of ligand L¹ can be used, based on Ru(II) or Ru(III) in Ru(II) or Ru(III) starting compound, for example 1.1 to 5 mol equivalents of L¹ per Ru(II) or Ru(III).

When carrying out the process according to the invention, water is formed in situ as by-product. It is preferred to separate off the water which is formed, also called water of reaction for short.

In one embodiment of the present invention, the water of reaction is separated off by separating it off with an azeotropic entrainer, for example one of the aforementioned solvents, in particular one of the aforementioned aromatic solvents. In a preferred variant, the procedure involves using alcohol of the general formula (II) as azeotropic entrainer since it has a miscibility gap with water in order to separate off, or remove azeotropically, water of reaction.

Preferably, the water of reaction is removed azeotropically during the reaction with the help of a water separator.

The process according to the invention can be carried out in a wide variety of reaction vessels in which liquid reactions, optionally with a gas space, can be carried out. Suitable reaction vessels are given for example in K. D. Henkel, “Reactor Types and Their Industrial Applications”, in Ullmann's Encyclopedia of Industrial Chemistry, 2005, Wiley-VCH Verlag GmbH & Co. KGaA, DOI: 10.1002/14356007.b04_{—}087, chapter 3.3 “Reactors for gas-liquid reactions”. Examples which may be mentioned are: stirred-tank reactors, tubular reactors and bubble-column reactors.

The process according to the invention can be carried out discontinuously, i.e. in batch mode, or continuously or semicontinuously with or without recycle. The average residence time of the reaction mass that is formed in the reaction vessel can be for example in the range from 15 minutes to 100 hours.

Without intending to give preference to a specific theory, it is thus plausible that the process according to the invention comprises essentially three reactions. Firstly, alcohol of the formula (II) is oxidatively dehydrogenated, specifically to give the aldehyde. An aldol condensation then takes place, followed by a reduction.

Implementation of the process according to the invention gives branched alcohol of the general formula (I)

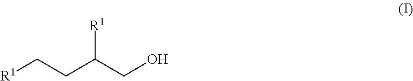

where the groups R¹ are different or identical and as defined above.

If a mixture of alcohol of the general formula (II) with one or more isomers is used as starting material, then a mixture of branched alcohols of the general formula (I) is usually obtained.

In one embodiment of the present invention, branched alcohol of the general formula (I.1)

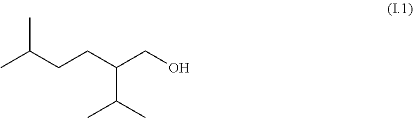

is obtained in a mixture with alcohol of the formula (Ia)

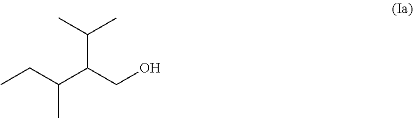

In another embodiment, branched alcohol of the formula (I.2)

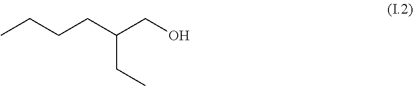

is obtained in a mixture with alcohol of the formula (Ia.2)

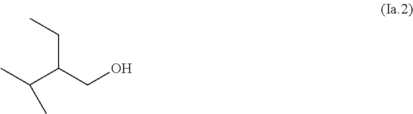

In one embodiment of the present invention, a further by-product obtained is esters, for example—if isoamyl alcohol is used as starting alcohol of the formula (I)—an ester of the formula

(CH₃)₂CH—(CH₂)—COO—(CH₂)₂—CH(CH₃)₂

In one embodiment of the present invention, the process according to the invention is carried out as far as complete conversion of alcohol of the general formula (II). In another embodiment, the reaction is carried out only to incomplete conversion, for example to 8 to 50 mol %, preferably to 30 mol %, followed by work-up.

Here, it is possible to recover and re-use Ru(II).

For the purpose of work-up, the procedure can involve, for example, separating off branched alcohol of the general formula (I) from unreacted alcohol of the general formula (II) and also from base and complex compound of Ru(II) by distillation. Complex compound of Ru(II) and base remains with any high-boiling components formed, for example trimerization product of alcohol of the general formula (II), in the bottom of the distillation and can be re-used. Unreacted alcohol of the general formula (II) can likewise be returned again to the reaction. The thermal separation of alcohol of the general formula (I) and also of optionally formed ester can take place for example by processes known per se, preferably in an evaporator or in a distillation unit, comprising evaporator and column(s), which usually has or have a plurality of trays or a packing or packing bodies.

By means of the process according to the invention it is possible to prepare branched alcohols of the formula (I) in good yield and very good purity. For their preparation, it is possible to start not only from pure alcohol of the general formula (II), but also to use isomer mixtures, for example those which can be obtained by fermentation or other biological degradation of saccharides, in particular so-called fusel oils.

The present invention further provides the use of catalysts comprising at least one Ru(II)-containing complex compound in which the Ru(II) has at least one ligand L¹ which is at least bidentate, where at least one coordination site of L¹ is a nitrogen atom, for preparing alcohols of the general formula (I)

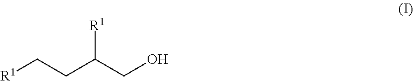

where the groups R¹ are different or identical and selected from C₂-C₃-alkyl, linear or branched, using at least one alcohol of the formula (II)

R¹—CH₂—CH₂—OH (II).

Here, the variables are as defined in more detail above.

In this connection, the use according to the invention is particularly preferred when it is desired to prepare branched alcohol of the formula (I) starting from alcohol of the formula (II), which is prepared on the basis of fusel oils, i.e. on the basis of so-called “bio-based isoamyl alcohols”.

In one variant of the use according to the invention, the Ru(II)-containing complex compound has at least one further ligand selected from CO, pseudohalides, organic carbonyl compounds, aromatics, olefins, phosphanes, hydride and halides. In a preferred variant, L¹ is selected from bidentate and tridentate ligands which coordinate with Ru(II) via nitrogen atoms and optionally via one or more carbene carbon atoms. Examples of particularly preferred ligands L¹ are given above.

The present invention is illustrated further by reference to working examples.

### WORKING EXAMPLES

General preliminary remarks:

Examples I.1 to I.11 were carried out under inert conditions (argon blanketing) in a 50 ml Schlenk flask with reflux condenser. Ruthenium(II) starting compound (0.05 mol % with respect to isoamyl alcohol), ligand L¹, the base (500 mg KOH; 9.7 mol %) and isoamyl alcohol (10 ml, (II.1)) were weighed into the Schlenk flask in a glove box. This gave a reaction mixture. The reaction mixture was stirred at 130° C. for 16 hours. Yield and selectivity of branched C₁₀-alcohol of the formula (I.1) was determined by gas chromatography (area %).

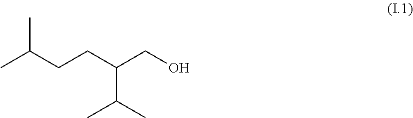

TABLE 1
Results of examples I.1 to I.11

| | | | Conver- | Selec- |
|---|---|---|---|---|
| | | | sion | tivity |
| Example | Ru(II) starting compound | L¹ | (II.1) | (I.1) |
| I.1 | [Ru(PPh₃)(H)₂(CO)] | (VII.1.2.a) | 31.8% | 92.5% |
| I.2 | [Ru(PPh₃)(H)₂(CO)] | (VI.1.a) | 29.5% | 91.9% |
| I.3 | [Ru(PPh₃)(H)₂(CO)] | (VII.1.1) | 8% | 75% |
| I.4 | [Ru(PPh₃)(H)₂(CO)] | (VII.1.2.a) | 15.9% | 88.1% |
| I.5 | [Ru(PPh₃)(H)₂(CO)] | (VI.1.b) | 33.0% | 85.1% |
| I.6 | [Ru(PPh₃)(H)₂(CO)] | (VII.1.3.a) | 15.0% | 74.0% |
| I.7 | [Ru(PPh₃)(H)₂(CO)] | (VII.1.3.b) | 10.2% | 100% |
| I.8 | [Ru(PPh₃)(H)₂(CO)] | (VII.1.3.c) | 23.0% | 39.6% |
| I.9 | [Ru(PPh₃)₃(H)(Cl)(CO)] | (VI.1.a) | 31.8% | 85.5% |
| I.10 | [Ru(PPh₃)₃(H)(Cl)(toluene)] | (VI.1.a) | 29.4% | 75.9% |
| I.11 | [Ru(PPh₃)₃(Cl)₂(CO)] | (VI.1.a) | 26.3% | 76.4% |

The following ligands L¹ were used:

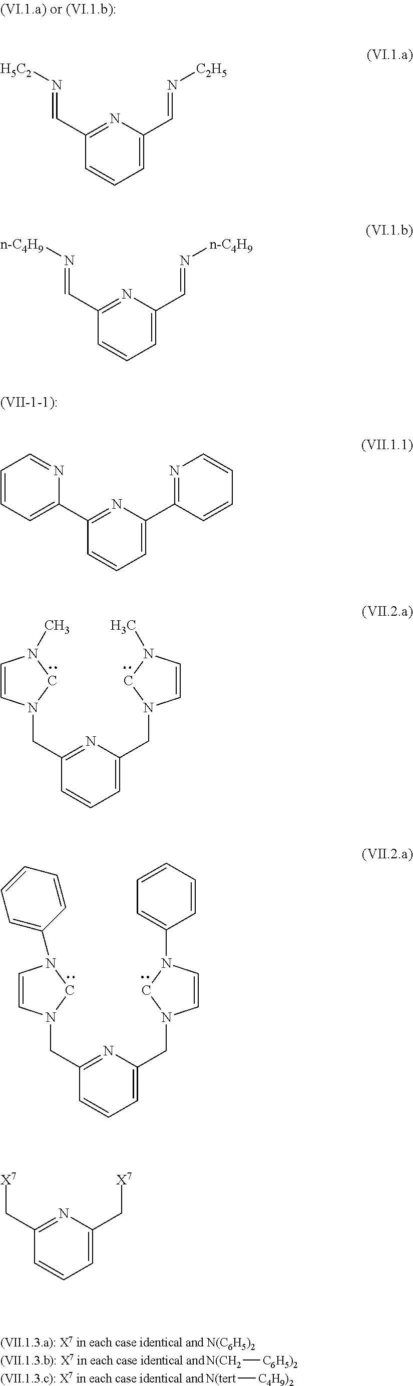

### Example II

Under inert conditions (glove box), 102 g (1.16 mol) of isoamyl alcohol, 5.0 g (89 mmol) of KOH, 130 mg (0.46 mmol) of [Ru(COD)(Cl)₂]₂ and 250 mg (1.35 mmol) of PPh₃ were weighed into a 250 ml three-neck flask. This gave a mixture which was covered with argon. The 250 ml three-neck flask was then equipped with a reflux condenser, the mixture was heated to 100° C. and stirred at 100° C. for two hours. Then, 120 mg (0.48 mmol) of the ligand (VI.1.a), dissolved in 2 ml of isoamyl alcohol, were added. The reaction mixture turned brown. The brown reaction mixture was then boiled at reflux over a period of 16 hours at an oil bath temperature of 170° C. using a water separator. The mixture was then also left to cool to room temperature. The gas chromatogram of the reaction mixture exhibited a conversion of isoamyl alcohol of 80.8% and a selectivity with respect to the alcohol of the formula (I.1) of 87.2%.

1. A process for preparing a branched alcohol of formula (I): 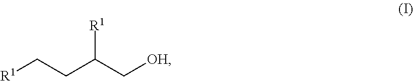
the process comprising reacting at least one alcohol of formula (II):
R¹—CH₂—CH₂—OH (II),
in a homogeneous phase, in the presence of at least one base and at least one Ru(II)-containing complex compound comprising at least one ligand L¹ which is at least bidentate, such that at least one coordination site of the ligand L¹ is a nitrogen atom,
wherein the groups R¹ are different or identical and represent a linear or branched C₂-C₃-alkyl.

2. The process according to claim 1, which occurs without adding a solvent which is different from the alcohol of formula (II).

3. The process according to claim 1, which occurs at temperatures in a range from 100 to 200° C.

4. The process according to claim 1, wherein R¹ is ethyl or isopropyl.

5. The process according to claim 1, wherein the at least one L¹ is selected from the group consisting of a bidentate ligand and a tridentate ligand, which coordinate with Ru(II) through one or more nitrogen atoms and optionally through one or more carbene carbon atoms.

6. The process according to claim 1, wherein the Ru(II)-containing complex compound further comprises at least one further ligand selected from the group consisting of CO, a pseudohalide, an organic carbonyl compound, an aromatic, an olefin, a phosphane, a hydride and a halide.

7. The process according to claim 1, wherein the at least one ligand L¹ is a compound of formula (III): 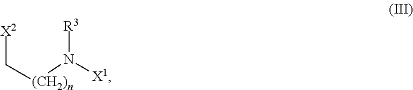
wherein:
R³ represents hydrogen or a C₁-C₅-alkyl;
n represents 0 or 1;
X¹ represents hydrogen, a C₁-C₅-alkyl, or (CH₂)ₙ₊₁—X²;
X² represents NR⁴R⁵, 2-pyridyl, or a imidazol-2-ylidenyl of the following formula: 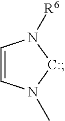
R⁴, R⁵ independently represent a C₁-C₁₀-alkyl, a C₃-C₁₀-cycloalkyl, a benzyl, or a phenyl, unsubstituted or mono- or polysubstituted with a C₁-C₃-alkyl; and
R⁶ represents a C₁-C₁₀-alkyl, a C₃-C₁₀-cycloalkyl, a benzyl and a phenyl, unsubstituted or mono- or polysubstituted with a C₁-C₃-alkyl.

8. The process according to claim 1, wherein the at least one ligand L¹ is a compound of formula (IV): 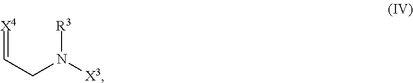
wherein:
R³ represents hydrogen, a C₁-C₁₀-alkyl, a C₃-C₁₀-cycloalkyl, a benzyl, or a phenyl;
X³ represents hydrogen, a C₁-C₅-alkyl or a CH₂═X⁴;
X⁴ represents NR⁴; and
R⁴ represents a C₁-C₁₀-alkyl, a C₃-C₁₀-cycloalkyl, a benzyl, or a phenyl, unsubstituted or mono- or polysubstituted with a C₁-C₃-alkyl.

9. The process according to claim 1, wherein the at least one ligand L¹ is a compound of formula (V): 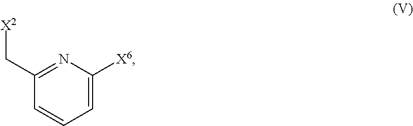
wherein:
X⁶ represents hydrogen, a C₁-C₅-alkyl, or a CH₂—X²;
X² represents NR⁴R⁵, a 2-pyridyl, or a imidazol-2-ylidenyl of the following formula: 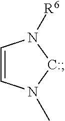
R⁴, R⁵ independently represent a C₁-C₁₀-alkyl, a C₃-C₁₀-cycloalkyl, a benzyl, or a phenyl, unsubstituted or mono- or polysubstituted with a C₁-C₃-alkyl; and
R⁶ represents a C₁-C₁₀-alkyl, a C₃-C₁₀-cycloalkyl, a benzyl, or a phenyl, unsubstituted or mono- or polysubstituted with a C₁-C₃-alkyl.

10. The process according to claim 1, wherein the least one ligand L¹ is a compound of formula (VI): 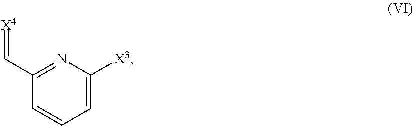
wherein:
X³ represents hydrogen, a C₁-C₅-alkyl, or CH₂═X⁴;
X⁴ independently represents N—R⁴;
R⁴ represents a C₁-C₁₀-alkyl, a C₃-C₈-cycloalkyl, a benzyl, or a phenyl, unsubstituted or mono- or polysubstituted with a C₁-C₃-alkyl.

11. The process according to claim 1, wherein the at least one ligand L¹ is a compound of formula (VII): 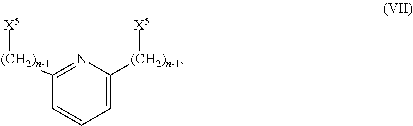
wherein:
n independently represents 0 or 1;
X⁵ is in each case identical and represents NR⁴R⁵, 2-pyridyl and a imidazol-2-ylidenyl of the following formula: 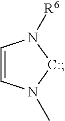
R⁴, R⁵ independently represent a C₁-C₁₀-alkyl, a C₃-C₁₀-cycloalkyl, a benzyl, or a phenyl, unsubstituted or mono- or polysubstituted with a C₁-C₃-alkyl; and
R⁶ represents a C₁-C₁₀-alkyl, a C₃-C₁₀-cycloalkyl, a benzyl, or a phenyl, unsubstituted or mono- or polysubstituted with a C₁-C₃-alkyl.

12. The process according to claim 1, wherein the Ru(II)-containing complex compound is formed in situ.

13. The process according to claim 1, wherein alcohol of the formula (II) is an azeotropic entrainer.

14. A catalyst comprising at least one Ru(II)-containing complex compound comprising at least one ligand L¹ which is at least bidentate, where at least one coordination site of the ligand L¹ is a nitrogen atom, said catalyst being suitable for preparing an alcohol of formula (I): 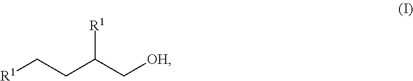
from at least one alcohol of formula (II):
R¹—CH₂—CH₂—OH (II),
wherein the groups R¹ are different or identical and represent a C₂-C₃-alkyl, linear or branched.

15. The use according to claim 14, wherein the Ru(II)-containing complex compound further comprises at least one further ligand selected from the group consisting of CO, a pseudohalide, an organic carbonyl compound, an aromatic, an olefin, a phosphane, a hydride and a halide.

16. The use according to claim 14, wherein at least one ligand L¹ is selected from the group consisting of a bidentate ligand and a tridentate ligand, which coordinate with Ru(II) through nitrogen atoms and optionally through one or more carbene carbon atoms.
